(19) United States
(12) Patent Application Publication
(10) Pub. No.: US 2026/0199117 A1
(43) Pub. Date: Jul. 16, 2026

(54) HIP ORTHOTIC WITH A REMOVABLE ABDUCTION PILLOW

(71) Applicants: Bryan E. Kilbey, DeFuniak Springs, FL (US); Jorge Chahla, DeFuniak Springs, FL (US)

(72) Inventors: Bryan E. Kilbey, DeFuniak Springs, FL (US); Jorge Chahla, DeFuniak Springs, FL (US)

(21) Appl. No.: 19/560,677

(22) Filed: Mar. 9, 2026

Related U.S. Application Data
(63) Continuation of application No. 17/979,224, filed on Nov. 2, 2022, now abandoned.
(63) Continuation-in-part of application No. 16/160,092, filed on Oct. 15, 2018.

(51) Int. Cl. A61F 5/01; A47C 20/02

(52) U.S. Cl. CPC A61F 5/0193; A47C 20/021; A47C 20/026

(57) ABSTRACT

An abductor pillow assembly having an abductor pillow removably attached to a panel assembly. The panel assembly has a waist panel, a central web and a thigh panel connected as one integrated panel. The panel assembly also includes a waist strap configured to encircle a user's waist and a thigh strap configured to encircle a first leg (upper thigh) of a user, The abductor pillow includes a main body and an abductor strap. The abductor strap passes through a slot in the main body and attaches at either end to the panel assembly, securing the abductor pillow to the user.

### BACKGROUND OF THE INVENTION

### 1. Field of the Invention

This invention relates to the field of medical products. More specifically, the invention comprises an abduction pillow assembly that is configured to attach to a portion of a hip brace to hold at least one of a patient's legs in a proper position to assist in a physician's pre- and post-operative protocol.

### 2. Description of the Related Art

Hip replacement surgery is often an open procedure requiring a relatively large incision. Significant tissue displacement is required during the preparation and insertion of an artificial joint. In recent years arthroscopic hip surgery has become increasingly common, and this has reduced the wound size and tissue disruption. Whether an open or closed procedure, it is generally important after such a surgery to control the motion of the hip joint, especially during sleep. Sleeping in the correct position is important to control pain and promote healing. Specifically, the patient's legs should be separated such that knees and ankles do not be cross the middle of the body. The legs should also remain straight.

Prior art devices only wrap around a user's legs. The devices are therefore less stable and do not adequately constrain the motion of the hip joint. The present invention addresses this concern by combining the use of a removable abduction pillow and the soft portion of a hip joint orthotic.

### BRIEF SUMMARY OF THE PRESENT INVENTION

The present invention provides an abductor pillow assembly that is affixable to a hip joint orthotic for use during sleep. The abductor pillow assembly includes a flexible panel assembly and an abductor pillow. The flexible panel assembly includes a waist strap that wraps around and is secured to a user's waist. The abductor pillow assembly includes an abductor strap that preferably passes through the main body of the abductor pillow and attaches the abductor pillow to the flexible panel assembly. The abductor pillow is adjustable in length and includes a series of cutout areas to aide in comfort of the user and effectiveness of the device.

The flexible panel assembly effectively wraps around the user's waist and leg. This two-point attachment creates stability in the hip joint. By connecting the abductor pillow to the flexible panel assembly (and therefore the waist of the user) the abductor pillow assembly prevents the user's motion while resting from shifting the abductor pillow in relation to the user's hip or legs.

### BRIEF DESCRIPTION OF THE SEVERAL VIEWS OF THE DRAWINGS

FIG. 1 is a perspective view showing the brace assembly used in the present invention.

FIG. 2 is a detailed view, showing an adjustable coupling.

FIG. 3 is a perspective view, showing the brace assembly installed on a user.

FIG. 4 is a perspective view, showing the flexible panel assembly lying flat.

FIG. 5 is a perspective view, showing the abductor pillow.

FIG. 6 is a perspective view, showing the abductor pillow.

FIG. 7 is a perspective view, showing the abductor pillow assembly in one embodiment.

FIG. 8 is a perspective view, showing the abductor pillow assembly installed on a user.

FIG. 9 is a perspective view, showing the abductor pillow assembly installed on a user.

### REFERENCE NUMERALS IN THE DRAWINGS

10 brace assembly
12 panel assembly
14 pivot mechanism
16 abductor pillow
18 main body
20 slot
22 series of scores
24 abductor strap
26 top portion
28 front
30 back
32 first side
34 second side
36 upper plate cover
38 waist panel
40 thigh panel
42 waist strap
44 thigh strap
45 lower thigh strap
46 top cutout
48 side cutout
50 base
52 central web
54 sheave assembly
56 adjustment tab
58 cord
60 sheave body
64 coupler
66 user
68 receiver
70 release button
72 hook panel
74 adjustable coupling
80 leg
84 hook panel
88 first end
90 second end

### DETAILED DESCRIPTION OF THE INVENTION

The following descriptions prefer to a preferred embodiment of the present invention. Upon reviewing these descriptions, those skilled in the art will envision many additional embodiments. Thus, the scope of the invention should be fixed by the claims rather than the specific embodiments described.

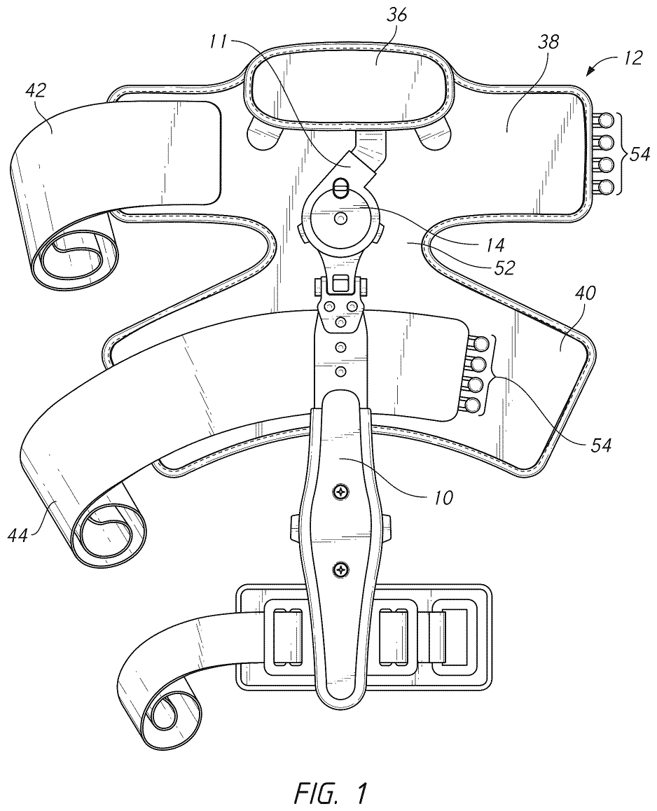

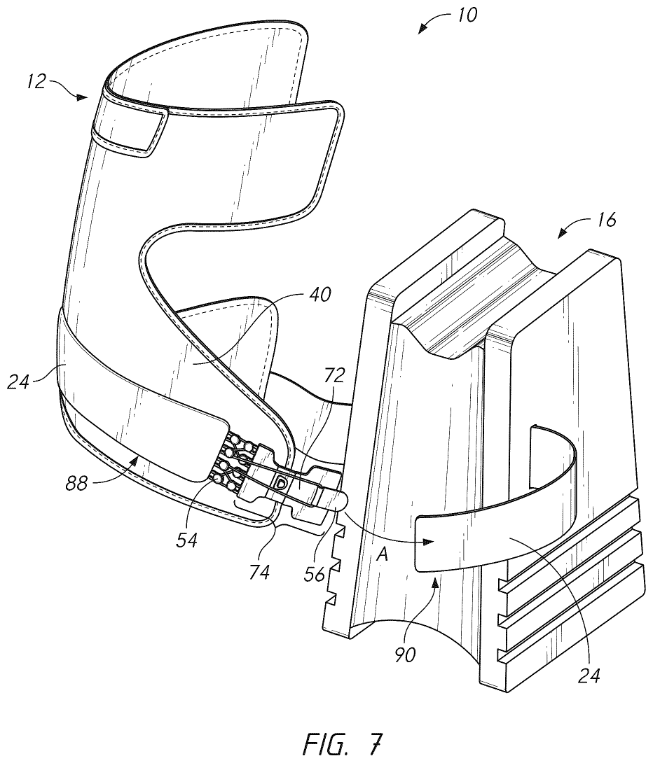

FIG. 7 depicts the major components of a preferred embodiment. Abductor pillow assembly 10 includes a flexible panel assembly 12, several belts and adjustment devices, and a removable abduction pillow 16. Flexible panel assembly 12 is preferably part of a convertible hip orthotic. This hip orthotic can be used in a “rigid” state (including a rigid motion-limiting brace assembly) and a “soft” state (in which the rigid brace assembly is removed). FIGS. 1-3 illustrate details of the hip orthotic in the rigid state. FIG. 1 shows rigid brace assembly 11 connected to flexible panel assembly 12. When in use, rigid brace assembly 11 runs roughly parallel to a user's torso and thigh. Rigid brace assembly 11 can be easily removed from panel assembly 12 for comfort while a user is resting (or at a later point in the recovery process when the use of a rigid brace assembly is deemed unnecessary).

Flexible panel assembly 12 preferably includes waist panel 38, thigh panel 40, and central web 52 (which links the waist panel and the thigh panel). Rigid brace assembly 11 includes a pivot mechanism 14 that lies roughly over the location of central web 52. Waist strap 42 has a first end secured to the left portion of waist panel 38 (“left” being understood with respect to the orientation shown in FIG. 1). A second end of the waist strap is free. The free end is passed around the user's waist and secured back to the assembly when the panel assembly is installed.

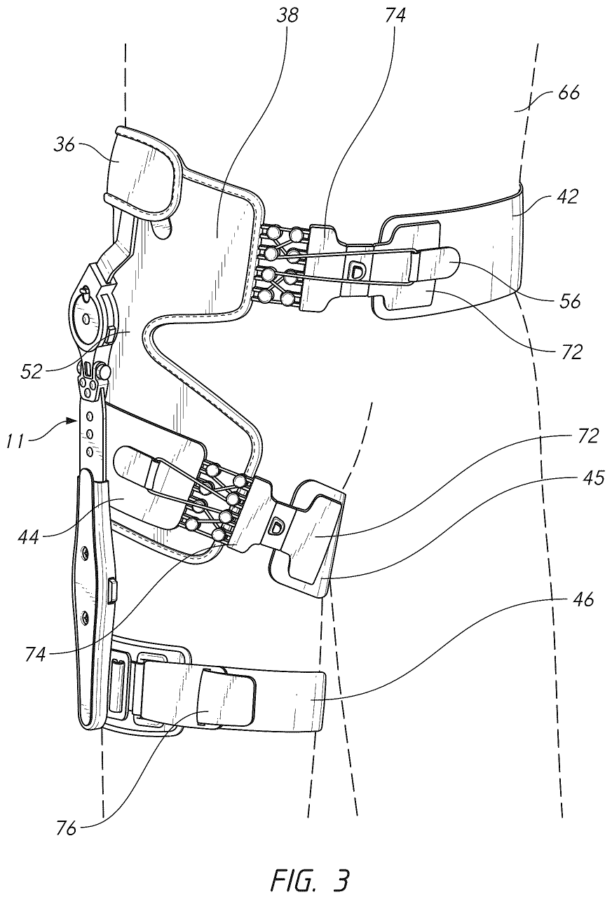

FIG. 3 shows the hip orthotic installed on user 66. Waist strap 42 passes around the user's waist and is adjustably secured using adjustable coupling 74. Upper thigh strap 44 passes around the user's upper thigh and is adjusted using a second adjustable coupling 74. Lower thigh strap 45 is attached to the lower portion of rigid brace assembly 11 and when rigid brace assembly 11 is removed from flexible panel assembly 12, lower thigh strap 45 remains with rigid brace assembly 11. When rigid brace assembly 11 is in use, lower thigh strap 45 is engaged through loops on the rigid brace assembly and assists in keeping the rigid brace assembly 11 in position.

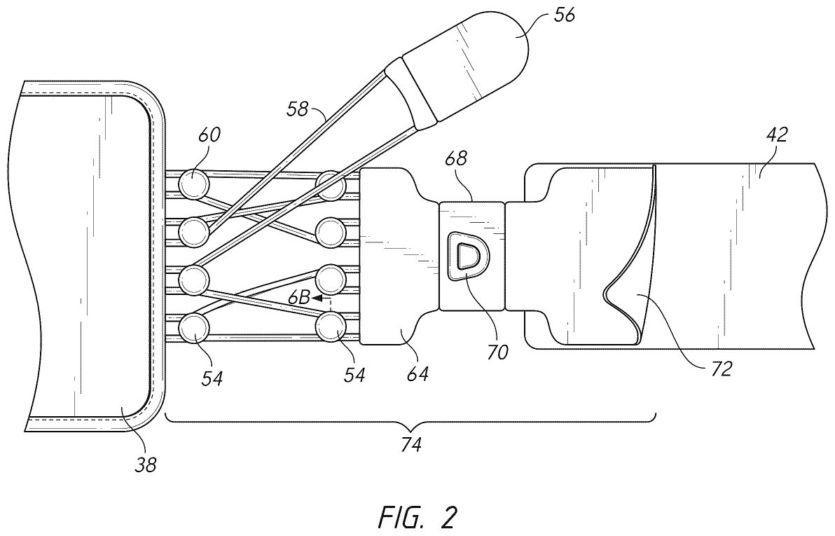

Many different types of adjustment features can be used for the encircling straps, and the invention is by no means limited to any particular type. Two adjustable couplings 74 are shown in the embodiment of FIG. 3. FIG. 2 shows one of these adjustable couplings 74 in greater detail. A sheave assembly 54 is attached to one end of waist panel 38. The sheave assembly 54 attached to panel 38 contains multiple sheave bodies 60. Coupler 64 likewise contains multiple sheave bodies 54. Coupler 64 is selectively engaged to receiver 68 by pressing release button 70. Receiver 68 is connected to hook panel 72, and hook panel 72 is connected to the free end of waist strap 42 by pressing the hook panel into the hook-compatible material on the waist strap at a desired location.

Cord 58 is threaded through the sheave bodies as shown. The two free ends of the cord are anchored to two of the sheave bodies on coupler 64. A loop of the cord passes through adjustment tab 56. The side of the adjustment tab facing away from the viewer is provides with a hook panel. This hook panel can be pressed into a hook-compatible covering on the outward-facing surface of waist strap 42. With this arrangement the user can pull adjustment tab 56 to tighten adjustable coupling 74 to a desired degree, then secure it by pressing tab 56 against waist strap 42 (as shown in FIG. 3).

In the embodiment depicted in FIG. 2, coupler 64 can be selectively disengaged from receiver 68 by pressing release button 70. The coupler can be reconnected to the receiver by sliding the two components together. When first donning the orthotic adjustment tab 56 can be left dangling. The user first connects the belt by snapping coupler 64 into receiver 68. The user then adds the desired level of tension by pulling adjustment tab 56 to the right and securing it in place.

Although this adjustment and attachment mechanism is shown and referenced, any known attachment and adjustment mechanism can be used to attach flexible panel assembly 12 to user and abductor pillow 16 for the present abductor pillow assembly.

FIG. 3 shows the orthotic with brace assembly 11 installed on user 66. Waist strap 42 is passed around the user's waist and secured using adjustable coupling 74. Upper thigh strap 44 is passed around the user's upper thigh and secured using adjustable coupling 97. Rigid brace assembly 11 is adjusted to the desired position and lower thigh strap 46 is then passed around the user's lower thigh. The lower thigh strap preferably includes loop covering on its inward and outward facing surfaces. Hook panel 76 is then pressed over the end of the belt and back on the body of the belt to secure it in position as shown.

Adjustment tab 56 is lifted and moved to adjust the desired tension of waist strap 42. Adjustment tab 56 over is lifted and moved to adjust the desired tension of upper thigh strap 44. A relatively low tension is applied with the position of adjustable tab 56 shown in FIG. 7. Hook panel 76 is lifted and moved to adjust the desired tension of lower thigh strap 46.

The reader will note that pivot mechanism 12 of brace assembly 11 is placed in line with the flexion/extension axis of the hip joint. The thin central web 52 of the panel assembly allows the panel assembly to bend with the pivot mechanism when the hip joint is flexed.

For use with the present abductor pillow assembly 10, the brace assembly 11 is easily removed from the flexible panel assembly 12. The flexible panel assembly 12 stays in place and the abductor pillow assembly 10 attaches thereto.

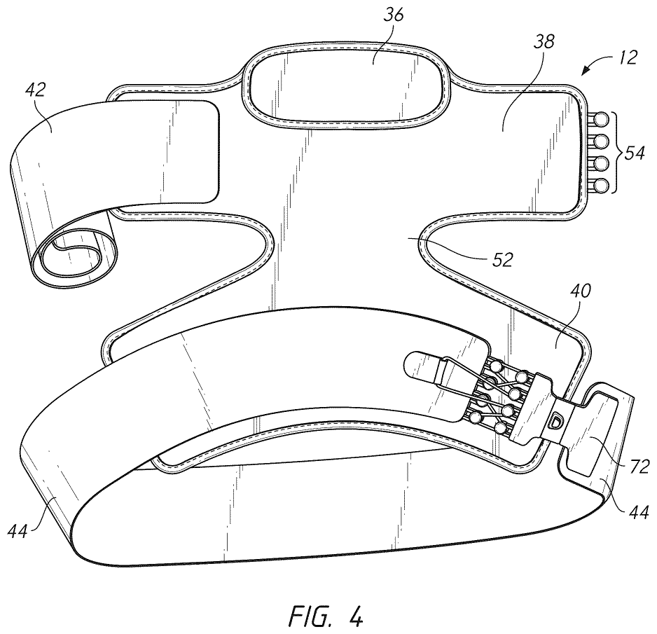

FIG. 4 illustrates flexible panel assembly 12 without rigid brace assembly 10. Flexible panel assembly 12 includes waist strap 42, waist panel 38, central web 52, thigh panel 40 and upper thigh strap 44. An optional upper plate cover 36 is included to cover a top portion of rigid brace 11 (shown in FIGS. 1 and 2). The outward facing surface of flexible panel assembly 12 including waist strap 42 are covered with loop material. Adjustable coupling 74 includes a hook panel 72 on its inward facing surface that attaches to waist strap 42 (as shown in FIGS. 2 and 3). Waist strap 42 can also include a hook panel on its inward facing surface to attach to waist panel 38 after wrapping around user's waist, rather than utilizing adjustable coupling 74 and sheave assembly 54. Upper thigh strap 44 includes hook panels to engage with loop material on thigh panel 40. Upper thigh strap 44 wraps around the user's upper thigh and affixes in the same manner as waist strap 42, described above. Upper thigh strap 44 is fully removable from thigh panel 40 and includes loop material on its outward facing surface.

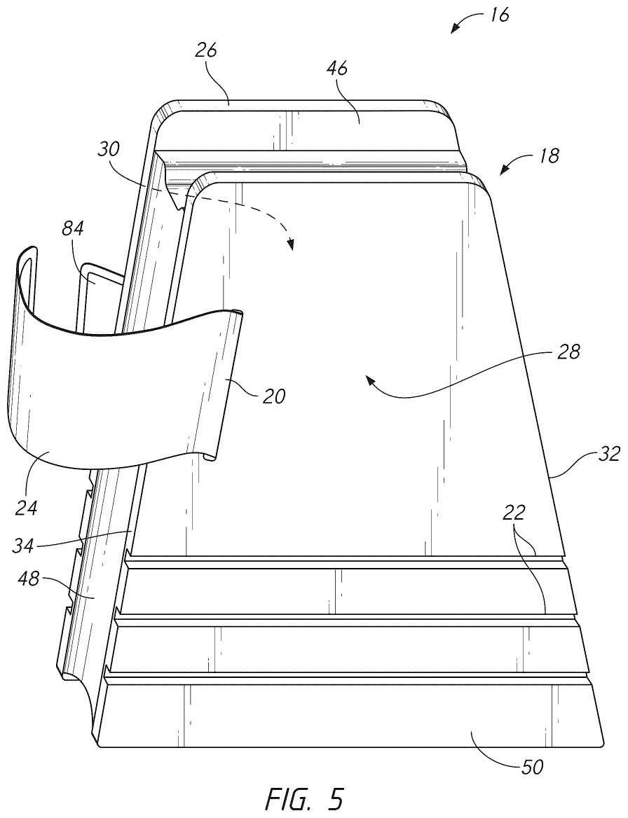

FIG. 5 is a perspective view of the abductor pillow 16. Abductor pillow 16 includes main body 18 and abductor strap 24. Main body 18 has a top portion 26, base 50, front 28, back 30, first side 32 and second side 34 (The directional terms such as “top,” “front,” and “back” refer to the orientation shown in FIG. 5. The abductor pillow can be used in more than one orientation and show these terms should not be viewed as limiting). Main body 18 is preferably fabricated using a molded foam such as polyurethane. The density and stiffness of the foam is selected so that it is flexible enough to deform to a desired extent when user attaches abductor pillow 16 to his or her legs. Top portion 26 of main body 18 includes a top cutout 46 for the male anatomy. First and second side (32, 34) include a curved side cutout 48 that accepts a user's leg on either side of main body 18. Abductor strap 24 passes through slot 20 on front 28 and back 30 of main body 18.

A series of scores 22 are notched into front 28 and back 30 of main body 18. Series of scores 22 can be used as a guide to easily cut to adjust the abductor pillow 16 to be progressively shorter in length for a user with shorter legs. An adjustable closure device is preferably provided to secure abductor strap 24 around a user's leg. In the embodiment shown, a hook panel 84 is provided on the inward facing surface of abductor strap 24. However, the reader will appreciate that another type of coupling—such as an adjustable coupling 74 having sheave assemblies 54—could be applied to thigh panel 40 to allow for attachment of abductor strap 24 to thigh panel 40 (or to the top of upper thigh strap 44), as shown and described with reference to FIG. 7.

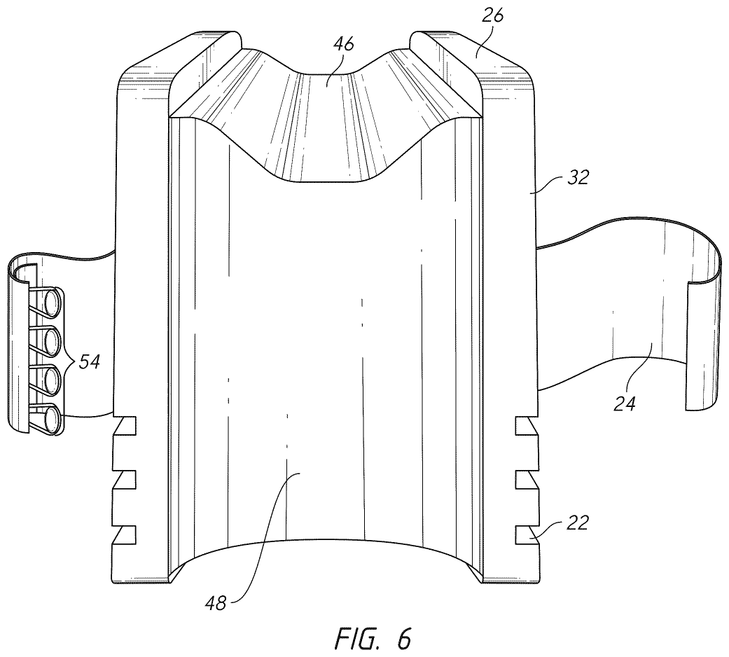

FIG. 6 shows a perspective view of first side 32 of main body 18. In this view, the profile of top cutout 46 along top portion 26 of main body 18 is clearer. The depth of series of scores 22 is illustrated. However, the reader will appreciate that series of scores 22 can be any depth so long as scores do not cause instability in the structure of main body 18. Abductor strap 24 extends outward from front 28 and back 30 of main body 18. Abductor strap 24 can be made of the same material as flexible panel assembly 12. In the version shown, a sheave assembly 54 is connected to an end of the abductor strap using including loop material on most of its surface with either a hook panel 84 on both ends (as shown in FIG. 5) or a sheave assembly 54 and hook panel 84 on only one end. Abductor strap 24 has a soft backing on most of its surface that prevents discomfort for the user.

One method of attaching flexible panel assembly 12 and abductor pillow 16 to each other to form abductor pillow assembly 10 is shown in FIG. 7. Abductor pillow 16 includes abductor strap 24 which has two ends. First end 88 of abductor strap 24 has an adjustable coupling 74 attached thereto. A hook panel on the side of the coupling facing away from the user can be pressed against the hook-compatible material on the other end of abductor strap 24 to secure the strap in a loop (as shown by arrow A). Tab 56 can then be used to tighten the strap as desired.

Upper thigh strap 44 is not shown in FIG. 7 for purposes of clarity. However, abductor strap 24 would likely overlap upper thigh strap 44 (In some embodiments the user will likely use the upper thigh strap as the abductor strap by passing a portion of it through slot 20 in abductor pillow 16). The flexible panel assembly 12 thereby maintains a set position on a user's leg, and abductor pillow 16 can be attached to flexible panel assembly 12, securing abductor pillow 16 in place between the user's legs.

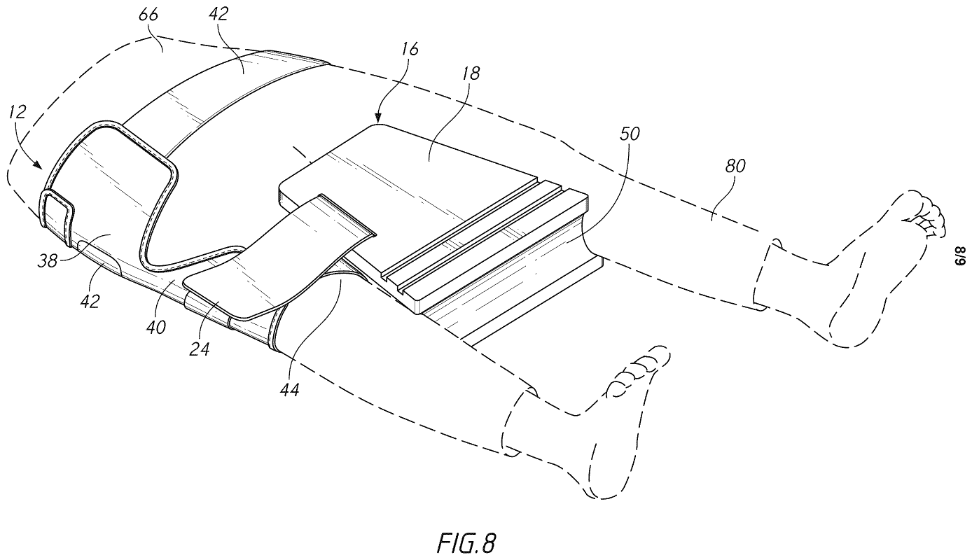

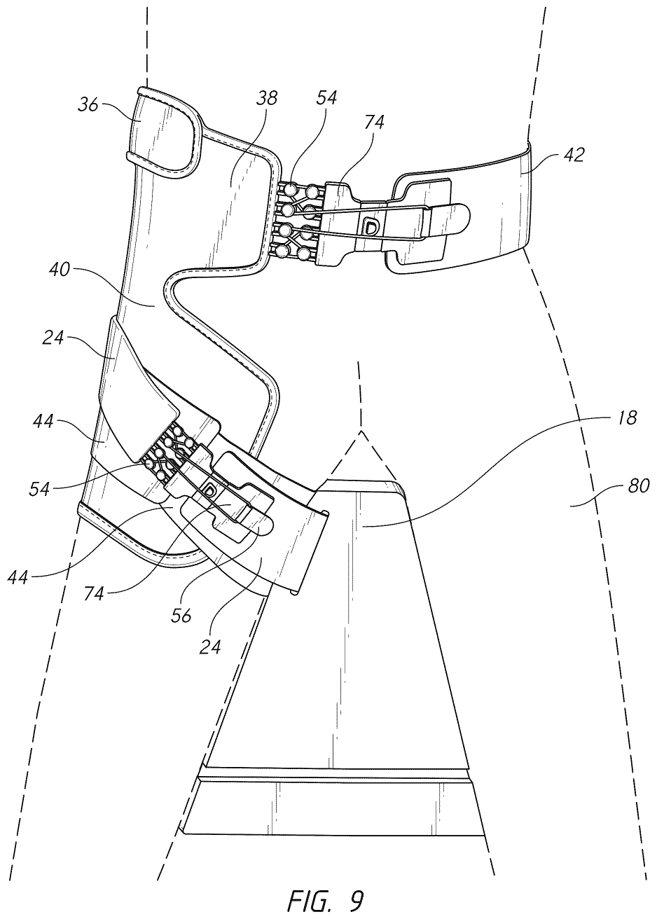

FIGS. 8 and 9 show the invention in an assembled state attached to a user. FIG. 8 illustrates the attachment of abductor pillow assembly 10 using straps that include hook panels alone. FIG. 9 illustrates the use of straps including the use of sheave assemblies and adjustable couplings, as described previously herein. The reader will appreciate that any attachment mechanism can be used. Additionally, it is preferable for all the straps employed to be adjustable in length over a wide range. One option is to provide straps that can be trimmed to length.

Returning to FIG. 8, to use the abductor pillow assembly 10, the user secures waist panel 38 to the affected hip and waist strap 42 is passed around the user's waist. Here, one end of waist strap 42 is sewn to waist panel 38 and the other end includes a hook panel to attach to the loop material on flexible panel assembly 12. The user can then affix a first end of upper thigh strap 44 to thigh panel 40 and pass strap around the user's leg, affixing the second end of upper thigh strap 44 to its first end. Once flexible panel assembly 12 is in position, the user places main body 18 of abductor pillow 16 between the user's legs. Side cutouts 48 (referenced in FIGS. 5 & 6) allow the user's legs to fit comfortably along the side of main body 18. Main body 18 holds the legs apart during rest so that the user's knees or ankles do not cross the centerline of the user's body.

For main body 18 to perform the function of maintaining the legs in a set position, abductor strap 24 is preferably secured to flexible panel assembly 12. The user wraps abductor strap 24 around leg and attaches a hook panel on first and second end of abductor strap 24 to the loop material on outer surface of thigh panel 40 or on outer surface of upper thigh strap 44. Top cutout 46 (shown best in FIG. 5) provides room for male anatomy in the configuration of FIG. 8.

FIG. 9 shows the abductor pillow assembly 10 attached to a user, utilizing straps (42, 44, 24) including sheave assemblies 54 and adjustable couplings 74. Waist strap 42 wraps around the user's waist and attaches to adjustable coupling 74. Adjustable coupling 74 and waist panel 38 are connected by sheave assemblies 54. The user can easily adjust the strap by tightening adjustment tab 56 on adjustable coupling 74. This secures waist panel 38 to waist of user. The user places main body 18 between legs 80. Once in place, user grasps abductor strap 24 which runs through main body 18 and attaches the first end of abductor strap 24 to loop material on the outer surface of thigh panel 40. The user next attaches the second end of abductor strap 24 to adjustable coupling 74. Once attached, the user can use adjustment tab 56 to tighten the two ends of abductor strap 24 together.

The connection of straps around hip and leg to a secure flexible panel assembly 12 acts to stabilize main body 18 of abductor pillow 16 in place such that the position of main body 18 is directly related to the position of the user's waist (or hip joint). The attachment of abductor pillow 16 to flexible panel assembly 12—which also attaches to the user's waist—effectively links the abductor pillow assembly to the user's leg and waist.

Although the preceding descriptions present considerable detail they should be properly viewed as illustrating embodiments of the present invention rather than limiting the scope of the invention. Many more embodiments following the same principles will occur to those skilled in the art. As an example, abductor pillow 16 could include a second abductor strap 24 that could wrap around the opposing leg of user and also affix to flexible panel assembly 12. Accordingly, the scope of the invention should be fixed by the following claims rather than by the examples given.

1. An abductor pillow assembly configured to attach to a user having a waist and a first and second leg, comprising: a panel assembly having:
a waist panel;
a thigh panel;
a central web between said waist panel and said thigh panel, said waist panel, thigh panel, and central web in combination forming a panel assembly;
a waist strap configured to encircle said waist of said user and secure said waist panel to said user;
an abductor pillow having:
a main body having a top portion, a front, a back, a first side, a second side and a base;
an abductor strap configured to encircle said leg of said user and secure said abductor pillow to said panel assembly;
a concave first cutout on said first side and parallel to said first side;
a concave second cutout on said second side and parallel to said second side; and
a top cutout in the firm of a transverse trough in said top portion running from said first side to said second side.

2. An abductor pillow assembly as recited in claim 1, wherein said panel assembly has a thigh strap configured to removably attach to said thigh panel at a first end and wrap around said first leg of said user and secure said thigh panel to said user on a second end.

3. An abductor pillow assembly as recited in claim 1, wherein said abductor pillow has a second abductor strap configured to encircle said second leg of said user.

4. An abductor pillow assembly as recited in claim 1, wherein said abductor strap has:
a first end having a hook panel,
a second end having a hook panel,
a length between said first end and said second end, and
wherein said hook panel on said first end removably attaches to said thigh panel, said length passes through said slot of said main body and said hook panel on said second end removably attaches to said thigh panel.

5. An abductor pillow assembly as recited in claim 4, wherein said attachment of said abductor pillow to said panel assembly connects said abductor pillow to said first leg and said waist of said user.

6. An abductor pillow assembly as recited in claim 2, wherein said abductor strap is configured to encircle said leg of said user and secure said abductor pillow to said thigh strap.

7. An abductor pillow assembly as recited in claim 1, wherein said abductor pillow assembly is configured to be fully removable from said panel assembly without removing said panel assembly from said user.

8. An abductor pillow assembly configured to attach to a user having a waist and a first and second leg, comprising:
a main body having a front, a back, a first side, a second side and a base;
at least one abductor strap configured to encircle said first leg of said user and secure said abductor pillow to said first leg;
a concave first cutout on said first side and parallel to said first side; and
a top cutout in the firm of a transverse trough in said top portion running from said first side to said second side.

9. An abductor pillow assembly as recited in claim 8, wherein said abductor pillow has a second abductor strap configured to encircle said second leg of said user.

10. An abductor pillow assembly as recited in claim 8, wherein said
at least one abductor strap has:
a first end having a hook panel,
a second end having a hook panel, and
a length between said first end and said second end.

11. An abductor pillow assembly as recited in claim 18, wherein said second side has a second side cutout configured to accept said second leg.
